(19) United States
(12) Patent Application Publication
(10) Pub. No.: US 2004/0018243 A1
(43) Pub. Date: Jan. 29, 2004

(54) Modulation of release from dry powder formulations

(75) Inventors: Sujit K. Basu, Cambridge, MA (US); Jeffrey S. Hrkach, Cambridge, MA (US); Giovanni Caponetti, Somerville, MA (US); Michael M. Lipp, Quincy, MA (US); Katharina Elbert, Cambridge, MA (US); Wen-I Li, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Advanced Inhalation Research, Inc., Cambridge, MA

(21) Appl. No.: 10/425,193

(22) Filed: Apr. 28, 2003

Related U.S. Application Data
(63) Continuation of application No. 09/644,736, filed on Aug. 23, 2000, now abandoned.
(60) Provisional application No. 60/150,742, filed on Aug. 25, 1999.

(51) Int. Cl. A61L 9/04; A61K 9/14; A61K 9/16; A61K 9/50

(52) U.S. Cl. 424/490; 424/46

(57) ABSTRACT

Particles which include a bioactive agent are prepared to have a desired matrix transition temperature. Delivery of the particles via the pulmonary system results in modulation of drug release from the particles. Sustained release of the drug can be obtained by forming particles which have a high matrix transition temperature, while fast release can be obtained by forming particles which have a low matrix transition temperature. Preferred particles include one or more phospholipids.

### RELATED APPLICATION(S)

This application is a continuation of U.S. application Ser. No. 09/644,736, filed Aug. 23, 2000, which claims the benefit of U.S. Provisional Application No. 60/150,742, filed Aug. 25, 1999. The entire teachings of the above application(s) are incorporated herein by reference.

### BACKGROUND OF THE INVENTION

Delivery via the pulmonary system can be self administered, circumvents painful injections as well as gastrointestinal complications or the unpleasant smell or taste associated with oral therapies.

Several compositions suitable for inhalation are currently available. For example, lipids-containing liposomes, pre-liposome powders and dehydrated liposomes for inhalation have been described as has been a bulk powder which includes a lipid and which, upon rehydration, spontaneously forms liposomes. Liposome formulations, however, often are unstable. Furthermore, liposomes, dehydrated liposomes as well as preliposome compositions generally require special manufacturing procedures or ingredients. Particles suitable for delivery via the pulmonary system which have a tap density of less than about 0.4 g/cm³ also have been described.

The release kinetics profile of a drug into the local and/or systemic circulation is an important treatment consideration. As known in the art, some medical indications require a sustained release of the drug. Several formulations suitable for inhalation and which also have controlled release properties have been described. In one example, particles having controlled release properties and a tap density of less than about 0.4 g/cm³ include a biocompatible, preferably a biodegradable polymer. Liposomal compositions with controlled release properties also are known.

Delivery of therapeutic agents via the pulmonary system can be used in systemic treatment protocols and also in the treatment of local lung disorders, such as asthma or cystic fibrosis. Albuterol sulfate, for example, is a β₂ agonist which can be used prophylactically to prevent asthmatic episodes. Extensive data and medical expertise in using albuterol sulfate in human patients has been accumulated. However, albuterol sulfate has a half life of only about 4 hours and longer lasting β₂ agonists are currently recommended in long term asthma management.

Therefore, a continued need exists for developing compositions which can deliver a medicament to the pulmonary system. A further need exists for developing compositions which can release the medicament at a desired release rate. A need also exists for developing compositions which reduce or eliminate drawbacks associated with some of the compositions currently available. Formulations which extend the protection afforded by a drug such as, for example, albuterol sulfate also are needed.

### SUMMARY OF THE INVENTION

The invention is generally directed to the pulmonary delivery of a bioactive agent. In particular, the invention is related to delivering via the pulmonary system particles which release the bioactive agent at a desired or targeted drug release rate. A fast release is obtained by forming particles which have a low matrix transition temperature, while delivery of particles which have a high matrix transition temperature results in a more sustained release of the bioactive agent. Particles having intermediate matrix transition temperatures, yielding intermediate drug release rates, also can be prepared.

In one embodiment of the invention, the particles include one or more phospholipids selected to have a desired phase transition temperature. In another embodiment of the invention, the particles have a tap density of less than about 0.4 g/cm³, preferably less than about 0.1 g/cm³.

The particles can be prepared by spray-drying methods. They are administered to the respiratory system of a subject using, for example, a dry powder inhaler.

The invention has numerous advantages. For example, particles having desired release kinetics can be prepared and delivered to the pulmonary system. The particles of the invention can be designed to have fast, intermediate or slow drug release rates and they can be preferentially delivered to a selected site of the pulmonary system. The particles include materials which may be the same or similar to surfactants endogenous to the lung and can be employed to deliver hydrophilic as well as hydrophobic medicaments via the pulmonary system. The particles of the invention are characterized by their matrix transition temperature and the matrix transition temperature can be used to design or optimize particle fromulations having a desired drug release profile.

Furthermore, the particles of the invention are not themselves liposomes, nor is it necessary for them to form lipisomes in the lung for their action. They can be formed under process conditions other than those generally required in fabricating liposomes or liposome-forming compositions.

### BRIEF DESCRIPTION OF THE DRAWINGS

FIG. 1 is a plot showing the first order release constants of particles of the invention which include albuterol sulfate formulations and unformulated albuterol sulfate.

FIG. 2 depicts the differential scanning calorimetry (DSC) thermograms of three formulations of albuterol sulfate.

FIG. 3 is a plot showing the correlation between the first order constants and matrix transition temperature for different albuterol sulfate formulations.

FIG. 4 depicts the differential scanning calorimetry (DSC) thermograms of two formulations of human serum albumin.

FIG. 5 shows the correlation between the first order release constants and matrix transition temperature for different albuterol sulfate formulations.

FIG. 6 is a schematic representation of particle behavior for particles having a matrix transition temperature which is less than about 37° Celsius (C) and for particles having a matrix transition temperature which is greater than about 37° C.

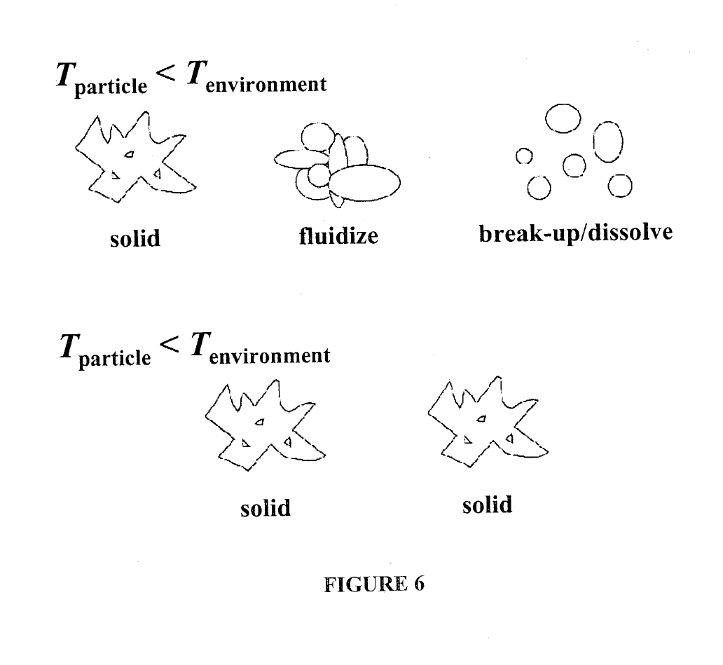

FIG. 7 is a plot showing the comparison of two albuterol sulfate formulations on carbachol-induced lung resistance in guinea pigs.

### DETAILED DESCRIPTION OF THE INVENTION

The invention is directed to the delivery of a bioactive agent via the pulmonary system. In particular, the invention is directed to particles which include a bioactive agent and which have desired drug release kinetics. In one embodiment of the invention, the particles, also referred to herein as powder, are in the form of a dry powder suitable for inhalation.

In a preferred embodiment of the invention, the bioactive agent is albuterol sulfate. Other therapeutic, prophylactic or diagnostic agents, also referred to herein as “bioactive agents”, “medicaments” or “drugs”, or combinations thereof, can be employed. Hydrophilic as well as hydrophobic drugs can be used.

Suitable bioactive agents include both locally as well as systemically acting drugs. Examples include but are not limited to synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which can, for instance, bind to complementary DNA to inhibit transcription, and ribozymes. The agents can have a variety of biological activities, such as vasoactive agents, neuroactive agents, hormones, anticoagulants, immunomodulating agents, cytotoxic agents, prophylactic agents, antibiotics, antivirals, antisense, antigens, antineoplastic agents and antibodies. In some instances, the proteins may be antibodies or antigens which otherwise would have to be administered by injection to elicit an appropriate response. Compounds with a wide range of molecular weight can be used, for example, between 100 and 500,000 grams or more per mole.

Proteins are defined as consisting of 100 amino acid residues or more; peptides are less than 100 amino acid residues. Unless otherwise stated, the term protein refers to both proteins and peptides. Examples include insulin and other hormones. Polysaccharides, such as heparin, can also be administered.

The particles may include a bioactive agent for local delivery within the lung, such as agents for the treatment of asthma, chronic obstructive pulmonary disease (COPD), emphysema, or cystic fibrosis, or for systemic treatment. For example, genes for the treatment of diseases such as cystic fibrosis can be administered, as can beta agonists steroids, anticholinergics, and leukotriene modifers for asthma. Other specific therapeutic agents include, but are not limited to, insulin, calcitonin, leuteinizing hormone releasing hormone (or gonadotropin-releasing hormone (“LHRH”)), granulocyte colony-stimulating factor (“G-CSF”), parathyroid hormone-related peptide, somatostatin, testosterone, progesterone, estradiol, nicotine, fentanyl, norethisterone, clonidine, scopolomine, salicylate, cromolyn sodium, salmeterol, formeterol, estrone sulfate, and valium.

Those therapeutic agents which are charged, such as most of the proteins, including insulin, can be administered as a complex between the charged therapeutic agent and a molecule of opposite charge. Preferably, the molecule of opposite charge is a charged lipid or an oppositely charged protein.

The particles can include any of a variety of diagnostic agents to locally or systemically deliver the agents following administration to a patient. Any biocompatible or pharmacologically acceptable gas can be incorporated into the particles or trapped in the pores of the particles using technology known to those skilled in the art. The term gas refers to any compound which is a gas or capable of forming a gas at the temperature at which imaging is being performed. In one embodiment, retention of gas in the particles is improved by forming a gas-impermeable barrier around the particles. Such barriers are well known to those of skill in the art.

Other imaging agents which may be utilized include commercially available agents used in positron emission tomography (PET), computer assisted tomography (CAT), single photon emission computerized tomography, x-ray, fluoroscopy, and magnetic resonance imaging (MRI).

Examples of suitable materials for use as contrast agents in MRI include the gadolinium chelates currently available, such as diethylene triamine pentacetic acid (DTPA) and gadopentotate dimeglumine, as well as iron, magnesium, manganese, copper and chromium.

Examples of materials useful for CAT and x-rays include iodine based materials for intravenous administration, such as ionic monomers typified by diatrizoate and iothalamate, non-ionic monomers such as iopamidol, isohexol, and ioversol, non-ionic dimers, such as iotrol and iodixanol, and ionic dimers, for example, ioxagalte.

Diagnostic agents can be detected using standard techniques available in the art and commercially available equipment.

The amount of therapeutic, prophylactic or diagnostic agent present in the particles can range from about 0.1 weight % to about 95% weight percent. Combinations of bioactive agents also can be employed. Particles in which the drug is distributed throughout a particle are preferred.

The particles of the invention have specific drug release properties. Drug release rates can be described in terms of the half-time of release of a bioactive agent from a formulation. As used herein the term “half-time” refers to the time required to release 50% of the initial drug payload contained in the particles. Fast drug release rates generally are less than 30 minutes and range from about 1 minute to about 60 minutes. Controlled release rates generally are longer than 2 hours and can range from about 1 hour to about several days.

Drug release rates can also be described in terms of release constants. The first order release constant can be expressed using one of the following equations:

M_{pw(t)}=M_{(∞)}*e^{-k*t} (1)

or,

M₍ₜ₎=M_{(∞)}*(1−e^{-k*t}) (2)

Where k is the first order release constant. M_{(∞)} is the total mass of drug in the drug delivery system, e.g. the dry powder, and M_{pw(t)} is drug mass remaining in the dry powders at time t. M₍ₜ₎ is the amount of drug mass released from dry powders at time t. The relationship can be expressed as:

M_{(∞)}=M_{pw(t)}+M₍ₜ₎ (3)

Equations (1), (2) and (3) may be expressed either in amount (i.e., mass) of drug released or concentration of drug released in a specified volume of release medium.

For example, Equation (2) may be expressed as:

C₍ₜ₎=C_{(∞)}*(1−e^{-k*t}) (4)

Where k is the first order release constant. C_{(∞)} is the maximum theoretical concentration of drug in the release medium, and C₍ₜ₎ is the concentration of drug being released from dry powders to the release medium at time t.

The ‘half-time’ or t_{50%} for a first order release kinetics is given by a well-know equation,

t_{50%} =0.693/k (5)

Drug release rates in terms of first order release constant and t_{50%} may be calculated using the following equations:

k=−ln(M_{pw(t)}/M_{(∞)})/t (6)

or,

k=−ln(M_{(∞)}−M₍ₜ₎)/M_{(∞)}/t (7)

Release rates of drugs from particles are controlled or optimized by adjusting the thermal properties or physical state transitions of the particles. The particles of the invention are characterized by their matrix transition temperature. As used herein, the term “matrix transition temperature” refers to the temperature at which particles are transformed from glassy or rigid phase with less molecular mobility to a more amorphorus, rubbery or molten state or fluid-like phase. As used herein, “matrix transition temperature” is the temperature at which the structural integrity of a particle is diminished in a manner which imparts faster release of drug from the particle. Above the matrix transition temperature, the particle structure changes so that mobility of the drug molecules increases resulting in faster release. In contrast, below the matrix transition temperature, the mobility of the drug particles is limited, resulting in a slower release. The “matrix transition temperature” can relate to different phase transition temperatures, for example, melting temperature (Tₘ), crystallization temperature (T_{c}) and glass transition temperature (T_{g}) which represent changes of order and/or molecular mobility within solids. The term “matrix transition temperature”, as used herein, refers to the composite or main transition temperature of the particle matrix above which release of drug is faster than below.

Experimentally, matrix transition temperatures can be determined by methods known in the art, in particular by differential scanning calorimetry (DSC). Other techniques to characterize the matrix transition behavior of particles or dry powders include synchrotron X-ray diffraction and freeze fracture electron micoscopy.

Matrix transition temperatures can be employed to fabricate particles having desired drug release kinetics and to optimize particle formulations for a desired drug release rate. Particles having a specified matrix transition temperature can be prepared and tested for drug release properties by in vitro or in vivo release assays, pharmacokinetic studies and other techniques known in the art. Once a relationship between matrix transition temperatures and drug release rates is established, desired or targeted release rates can be obtained by forming and delivering particles which have the corresponding matrix transition temperature. Drug release rates can be modified or optimized by adjusting the matrix transition temperature of the particles being administered.

The particles of the invention include one or more materials which, alone or in combination, promote or impart to the particles a matrix transition temperature that yields a desired or targeted drug release rate. Properties and examples of suitable materials or combinations thereof are further described below. For example, to obtain a rapid release of a drug, materials, which, when combined, result in a low matrix transition temperatures, are preferred. As used herein, “low transition temperature” refers to particles which have a matrix transition temperature which is below or about the physiological temperature of a subject. Particles possessing low transition temperatures tend to have limited structural integrity and be more amorphous, rubbery, in a molten state, or fluid-like.

Without wishing to be held to any particular interpretation of a mechanism of action, it is believed that, for particles having low matrix transition temperatures, the integrity of the particle matrix undergoes transition within a short period of time when exposed to body temperature (typically around 37° C.) and high humidity (approaching 100% in the lungs) and that the components of these particles tend to possess high molecular mobility allowing the drug to be quickly released and available for uptake.

In contrast, for the sustained release of a drug, materials, which, when combined, result in high matrix transition temperatures, are preferred. As used herein, “high transition temperature” refers to particles which have a matrix transition temperature which is higher than the physiological temperature of a subject. Particles possessing high transition temperatures will possess more structural integrity.

Without wishing to be held to any particular interpretation of a mechanism of action, it is believed that, for particles having high matrix transition temperatures, the integrity of the particle matrix can be maintained for longer periods at body temperature and high humidity resulting in slower particle melting, dissolution or erosion, a lower molecular mobility, and a slower drug release from the particle and a prolonged subsequent drug uptake and/or action.

Designing and fabricating particles with a mixture of materials having high phase transition temperatures and low phase transition temperatures can be employed to modulate or adjust matrix transition temperatures of resulting particles and corresponding release profiles for a given drug.

Combining appropriate amount of materials to produce particles having a desired transition temperature can be determined experimentally, for example by forming particles having varying proportions of the desired materials, measuring the matrix transition temperatures of the mixtures (for example by DSC), selecting the combination having the desired matrix transition temperature and, optionally, further optimizing the proportions of the materials employed.

Miscibility of the materials in one another also can be considered. Materials which are miscible in one another tend to yield an intermediate overall matrix transition temperature, all other things being equal. On the other hand, materials which are immiscible in one another tend to yield an overall matrix transition temperature that is governed either predominantly by one component or may result in biphasic release properties.

In a preferred embodiment, the particles include one or more phospholipids. The phospholipid or combination of phospholipids is selected to impart specific drug release properties to the particles. Phospholipids suitable for pulmonary delivery to a human subject are preferred. In one embodiment, the phospholipid is endogenous to the lung. In another embodiment, the phospholipid is non-endogenous to the lung.

The phospholipid can be present in the particles in an amount ranging from about 1 to about 99 weight %. Preferably, it can be present in the particles in an amount ranging from about 10 to about 80 weight %.

Examples of phospholipids include, but are not limited to, phosphatidic acids, phosphatidylcholines, phosphatidylethanolamines, phosphatidylglycerols, phosphatidylserines, phosphatidylinositols or a combination thereof. Modified phospholipids for example, phospholipids having their head group modified, e.g., alkylated or polyethylene glycol (PEG)-modified, also can be employed.

In a preferred embodiment, the matrix transition temperature of the particles is related to the phase transition temperature, as defined by the melting temperature (Tₘ), the crystallization temperature (T_{c}) and the glass transition temperature (T_{g}) of the phospholipid or combination of phospholipids employed in forming the particles. Tₘ, T_{c} and T_{g} are terms known in the art. For example, these terms are discussed in Phospholipid Handbook (Gregor Cevc, editor, 1993) Marcel-Dekker, Inc.

Phase transition temperatures for phospholipids or combinations thereof can be obtained from the literature. Sources listing phase transition temperature of phospholipids is, for instance, the Avanti Polar Lipids (Alabaster, Ala.) Catalog or the Phospholipid Handbook (Gregor Cevc, editor, 1993) Marcel-Dekker, Inc. Small variations in transition temperature values listed from one source to another may be the result of experimental conditions such as moisture content.

Experimentally, phase transition temperatures can be determined by methods known in the art, in particular by differential scanning calorimetry. Other techniques to characterize the phase behavior of phospholipids or combinations thereof include synchrotron X-ray diffraction and freeze fracture electron micoscopy.

Combining the appropriate amounts of two or more phospholipids to form a combination having a desired phase transition temperature is described, for example, in the Phospholipid Handbook (Gregor Cevc, editor, 1993) Marcell-Dekker, Inc. A particular example also is presented below as Example 3. Miscibilities of phospholipids in one another may be found in the Avanti Polar Lipids (Alabaster, Ala.) Catalog.

The amounts of phospholipids to be used to form particles having a desired or targeted matrix transition temperature can be determined experimentally, for example by forming mixtures in various proportions of the phospholipids of interest, measuring the transition temperature for each mixture, and selecting the mixture having the targeted transition temperature. The effects of phospholipid miscibility on the matrix transition temperature of the phospholipid mixture can be determined by combining a first phospholipid with other phospholipids having varying miscibilities with the first phospholipid and measuring the transition temperature of the combinations.

Combinations of one or more phospholipids with other materials also can be employed to achieve a desired matrix transition temperature. Examples include polymers and other biomaterials, such as, for instance, lipids, sphingolipids, cholesterol, surfactants, polyaminoacids, polysaccharides, proteins, salts and others. Amounts and miscibility parameters selected to obtain a desired or targeted matrix transition temperatures can be determined as described above.

In general, phospholipids, combinations of phospholipids, as well as combinations of phospholipids with other materials, which yield a matrix transition temperature no greater than about the physiological body temperature of a patient, are preferred in fabricating particles which have fast drug release properties. Such phospholipids or phospholipid combinations are referred to herein as having low transition temperatures. Examples of suitable low transition temperature phospholipids are listed in Table 1. Transition temperatures shown are obtained from the Avanti Polar Lipids (Alabaster, Ala.) Catalog.

TABLE 1

| | | Transition |
|---|---|---|
| | | Tem- |
| | Phospholipids | perature |
| 1 | 1,2-Dilauroyl-sn-glycero-3-phosphocholine (DLPC) | −1° C. |
| 2 | 1,2-Ditridecanoyl-sn-glycero-3-phosphocholine | 14° C. |
| 3 | 1,2-Dimyristoyl-sn-glycero-3-phosphocholine (DMPC) | 23° C. |
| 4 | 1,2-Dipentadecanoyl-sn-glycero-3-phosphocholine | 33° C. |
| 5 | 1,2-Dipalmitoyl-sn-glycero-3-phosphocholine (DPPC) | 41° C. |
| 6 | 1-Myristoyl-2-palmitoyl-sn-glycero-3-phosphocholine | 35° C. |
| 7 | 1-Myristoyl-2-stearoyl-sn-glycero-3-phosphocholine | 40° C. |
| 8 | 1-Palmitoyl-2-myristoyl-sn-glycero-3-phosphocholine | 27° C. |
| 9 | 1-Stearoyl-2-myristoyl-sn-glycero-3-phosphocholine | 30° C. |
| 10 | 1,2-Dilauroyl-sn-glycero-3-phosphate (DLPA) | 31° C. |
| 11 | 1,2-Dimyristoyl-sn-glycero-3-[phospho-L-serine] | 35° C. |
| 12 | 1,2-Dimyristoyl-sn-glycero-3-[phospho-rac-(1-glycerol)] | 23° C. |
| | (DMPG) | |
| 13 | 1,2-Dipalmitoyl-sn-glycero-3-[phospho-rac-(1-glycerol)] | 41° C. |
| | (DPPG) | |
| 14 | 1,2-Dilauroyl-sn-glycero-3-phosphoethanolamine (DLPE) | 29° C. |

Phospholipids having a head group selected from those found endogenously in the lung, e.g., phosphatidylcholine, phosphatidylethanolamines, phosphatidylglycerols, phosphatidylserines, phosphatidylinositols or a combination thereof are preferred.

The above materials can be used alone or in combinations. Other phospholipids which have a phase transition temperature no greater than a patient's body temperature, also can be employed, either alone or in combination with other phospholipids or materials.

In general, phospholipids, combinations of phospholipids, as well as combinations of phospholipids with other materials, which have a phase transition temperature greater than about the physiological body temperature of a patient, are preferred in forming slow release particles. Such phospholipids or phospholipid combinations are referred to herein as having high transition temperatures.

Examples of suitable high transition temperature phospholipids are shown in Table 2. Transition temperatures shown are obtained from the Avanti Polar Lipids (Alabaster, Ala.) Catalog.

TABLE 2

| | | Transition |
|---|---|---|
| | | Tem- |
| | Phospholipids | perature |
| 1. | 1,2-Diheptadecanoyl-sn-glycero-3-phosphocholine | 48° C. |
| 2. | 1,2-Distearoyl-sn-glycero-3-phosphocholine (DSPC) | 55° C. |
| 3. | 1-Palmitoyl-2-stearoyl-sn-glycero-3-phosphocholine | 49° C. |
| 4. | 1,2-Dimyristoyl-sn-glycero-3-phosphate (DMPA) | 50° C. |
| 5. | 1,2-Dipalmitoyl-sn-glycero-3-phosphate (DPPA) | 67° C. |
| 6. | 1,2-Dipalmitoyl-sn-glycero-3-[phospho-L-serine] | 54° C. |
| 7. | 1,2-Distearoyl-sn-glycero-3-[phospho-L-serine] | 68° C. |
| 8. | 1,2-Distearoyl-sn-glycero-3-[phospho-rac-(1-glycerol)] | 55° C. |
| | (DSPG) | |
| 9. | 1,2-Dimyristoyl-sn-glycero-3-phosphoethanolamine | 50° C. |
| | (DMPE) | |
| 10. | 1,2-Dipalmitoyl-sn-glycero-3-phosphoethanolamine | 63° C. |
| | (DPPE) | |
| 11. | 1,2-Distearoyl-sn-glycero-3-phosphoethanolamine | 74° C. |
| | (DSPE) | |

Phospholipids having a head group selected from those found endogenously in the lung, e.g., phosphatidylcholine, phosphatidylethanolamines, phosphatidylglycerols, phosphatidylserines, phosphatidylinositols or a combination thereof are preferred.

The above materials can be used alone or in combinations. Other phospholipids which have a phase transition temperature greater than a patient's body temperature also can be employed, either alone or in combination with other phospholipids or materials.

To obtain a targeted or desired release rate of a biologically active agent, for example an intermediate drug release rate, particles also can be manufactured by combining at least one phospholipid having a low transition temperature with at least one phospholipid having a high transition temperature.

Suitable methods of preparing and administering particles which include phospholipids, are described in U.S. Pat. No. 5,855,913, issued on Jan. 5, 1999 to Hanes et al. and in U.S. Pat. No. 5,985,309, issued on Nov. 16, 1999 to Edwards et al. The teachings of both are incorporated herein by reference in their entirety.

The particles can include one or more additional materials. Optionally, at least one of the one or more additional materials also is selected in a manner such that its combination with the phospholipids discussed above results in particles having a matrix transition temperature which results in the targeted or desired drug release rate.

In one embodiment of the invention, the particles further include polymers. Biocompatible or biodegradable polymers are preferred. Such polymers are described, for example, in U.S. Pat. No. 5,874,064, issued on Feb. 23, 1999 to Edwards et al., the teachings of which are incorporated herein by reference in their entirety.

In another embodiment, the particles include a surfactant other than one of the phospholipids described above. As used herein, the term “surfactant” refers to any agent which preferentially absorbs to an interface between two immiscible phases, such as the interface between water and an organic polymer solution, a water/air interface or organic solvent/air interface. Surfactants generally possess a hydrophilic moiety and a lipophilic moiety, such that, upon absorbing to microparticles, they tend to present moieties to the external environment that do not attract similarly-coated particles, thus reducing particle agglomeration. Surfactants may also promote absorption of a therapeutic or diagnostic agent and increase bioavailability of the agent.

Suitable surfactants which can be employed in fabricating the particles of the invention include but are not limited to hexadecanol; fatty alcohols such as polyethylene glycol (PEG); polyoxyethylene-9-lauryl ether; a surface active fatty acid, such as palmitic acid or oleic acid; glycocholate; surfactin; a poloxomer; a sorbitan fatty acid ester such as sorbitan trioleate (Span 85); and tyloxapol.

The surfactant can be present in the particles in an amount ranging from about 0 to about 60 weight %. Preferably, it can be present in the particles in an amount ranging from about 5 to about 50 weight %.

In yet another embodiment of the invention, the particles also include an amino acid. Suitable amino acids include naturally occurring and non-naturally occurring hydrophobic amino acids. Some suitable naturally occurring hydrophobic amino acids, include but are not limited to, leucine, isoleucine, alanine, valine, phenylalanine, glycine and tryptophan. Combinations of hydrophobic amino acids can also be employed Non-naturally occurring amino acids include, for example, beta-amino acids. Both D, L configurations and racemic mixtures of hydrophobic amino acids can be employed. Suitable hydrophobic amino acids can also include amino acid derivatives or analogs. As used herein, an amino acid analog includes the D or L configuration of an amino acid having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight chained, branched or cyclic C1-C8 hydrocarbons which are completely saturated, which contain one or two heteroatoms such as nitrogen, oxygen or sulfur and/or which contain one or more units of unsaturation. Aromatic groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridintyl.

Suitable substituents on an aliphatic, aromatic or benzyl group include —OH, halogen (—Br, —Cl, —I and —F) —O(aliphatic, substituted aliphatic, benzyl, substituted benzyl, aryl or substituted aryl group), —CN, —NO₂, —COOH, —NH₂, —NH(aliphatic group, substituted aliphatic, benzyl, substituted benzyl, aryl or substituted aryl group), —N(aliphatic group, substituted aliphatic, benzyl, substituted benzyl, aryl or substituted aryl group)₂, —COO(aliphatic group, substituted aliphatic, benzyl, substituted benzyl, aryl or substituted aryl group), —CONH₂, —CONH(aliphatic, substituted aliphatic group, benzyl, substituted benzyl, aryl or substituted aryl group)), —SH, —S(aliphatic, substituted aliphatic, benzyl, substituted benzyl, aromatic or substituted aromatic group) and —NH—C(═NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more substituents. Modifying an amino acid substituent can increase, for example, the lypophilicity or hydrophobicity of natural amino acids which are hydrophilic.

A number of the suitable amino acids, amino acids analogs and salts thereof can be obtained commercially. Others can be synthesized by methods known in the art. Synthetic techniques are described, for example, in Green and Wuts, “Protecting Groups in Organic Synthesis”, John Wiley and Sons, Chapters 5 and 7, 1991.

Hydrophobicity is generally defined with respect to the partition of an amino acid between a nonpolar solvent and water. Hydrophobic amino acids are those acids which show a preference for the nonpolar solvent. Relative hydrophobicity of amino acids can be expressed on a hydrophobicity scale on which glycine has the value 0.5. On such a scale, amino acids which have a preference for water have values below 0.5 and those that have a preference for nonpolar solvents have a value above 0.5. As used herein, the term hydrophobic amino acid refers to an amino acid that, on the hydrophobicity scale has a value greater or equal to 0.5, in other words, has a tendency to partition in the nonpolar acid which is at least equal to that of glycine.

Examples of amino acids which can be employed include, but are not limited to: glycine, proline, alanine, cysteine, methionine, valine, leucine, tyrosine, isoleucine, phenylalanine, tryptophan. Preferred hydrophobic amino acids include leucine, isoleucine, alanine, valine, phenylalanine, glycine and tryptophan. Combinations of hydrophobic amino acids can also be employed. Furthermore, combinations of hydrophobic and hydrophilic (preferentially partitioning in water) amino acids, where the overall combination is hydrophobic, can also be employed. Combinations of one or more amino acids and one or more phospholipids or surfactants can also be employed.

The amino acid can be present in the particles of the invention in an amount of at least 60 weight %. Preferably, the amino acid can be present in the particles in an amount ranging from about 5 to about 30 weight %. The salt of a hydrophobic amino acid can be present in the particles of the invention in an amount of at least 60 weight %. Preferably, the amino acid salt is present in the particles in an amount ranging from about 5 to about 30 weight %. Methods of forming and delivering particles which include an amino acid are described in U.S. patent application Ser. No. 09/382,959, filed on Aug. 25, 1999, entitled Use of Simple Amino Acids to Form Porous Particles During Spray Drying and U.S. patent application entitled Use of Simple Amino Acids to Form Porous Particles, filed concurrently herewith under Attorney Docket No. 2685.1004-001; the teachings of both are incorporated herein by reference in their entirety.

In a further embodiment of the invention, the particles also include a carboxylate moiety and a multivalent metal salt. Such compositions are described in U.S. Provisional Application 60/150,662, entitled Formulation for Spray-Drying Large Porous Particles, filed on Aug. 25, 1999 and U.S. patent application entitled Formulation for Spray-Drying Large Porous Particles, filed concurrently herewith under Attorney Docket No. 2685.2010-001; the teachings of which are incorporated herein by reference in their entirety. In a preferred embodiment, the particles include sodium citrate and calcium chloride.

The particles can also include other materials such as, for example, buffer salts, dextran, polysaccharides, lactose, trehalose, cyclodextrins, proteins, peptides, polypeptides, fatty acids, fatty acid esters, inorganic compounds, phosphates.

In a preferred embodiment, the particles of the invention have a tap density less than about 0.4 g/cm³. Particles which have a tap density of less than about 0.4 g/cm³ are referred to herein as “aerodynamically light particles”. More preferred are particles having a tap density less than about 0.1 g/cm³. Tap density can be measured by using instruments known to those skilled in the art such as the Dual Platform Microprocessor Controlled Tap Density Tester (Vankel, N.C.) or a GeoPyc™ instrument (Micrometrics Instrument Corp., Norcross, Ga. 30093). Tap density is a standard measure of the envelope mass density. Tap density can be determined using the method of USP Bulk Density and Tapped Density, United States Pharmacopia convention, Rockville, Md., 10^{th} Supplement, 4950-4951, 1999. Features which can contribute to low tap density include irregular surface texture and porous structure.

The envelope mass density of an isotropic particle is defined as the mass of the particle divided by the minimum sphere envelope volume within which it can be enclosed. In one embodiment of the invention, the particles have an envelope mass density of less than about 0.4 g/cm³.

Aerodynamically light particles have a preferred size, e.g., a volume median geometric diameter (VMGD) of at least about 5 microns (μm). In one embodiment, the VMGD is from about 5 μm to about 30 μm. In another embodiment of the invention, the particles have a VMGD ranging from about 9 μm to about 30 μm. In other embodiments, the particles have a median diameter, mass median diameter (MMD), a mass median envelope diameter (MMED) or a mass median geometric diameter (MMGD) of at least 5 μm, for example from about 5 μm to about 30 μm.

The diameter of the particles, for example, their VMGD, can be measured using an electrical zone sensing instrument such as a Multisizer IIe, (Coulter Electronic, Luton, Beds, England), or a laser diffraction instrument (for example Helos, manufactured by Sympatec, Princeton, N.J.). Other instruments for measuring particle diameter are well known in the art. The diameter of particles in a sample will range depending upon factors such as particle composition and methods of synthesis. The distribution of size of particles in a sample can be selected to permit optimal deposition within targeted sites within the respiratory tract.

Aerodynamically light particles preferably have “mass median aerodynamic diameter” (MMAD), also referred to herein as “aerodynamic diameter”, between about 1 μm and about 5 μm. In one embodiment of the invention, the MMAD is between about 1 μm and about 3 μm. In another embodiment, the MMAD is between about 3 μm and about 5 μm.

Experimentally, aerodynamic diameter can be determined by employing a gravitational settling method, whereby the time for an ensemble of particles to settle a certain distance is used to infer directly the aerodynamic diameter of the particles. An indirect method for measuring the mass median aerodynamic diameter (MMAD) is the multi-stage liquid impinger (MSLI).

The aerodynamic diameter, dₐₑᵣ, can be calculated from the equation:

dₐₑᵣ=d_{g}√ρₜₐₚ

where d_{g} is the geometric diameter, for example the MMGD and ρ is the powder density.

Particles which have a tap density less than about 0.4 g/cm³, median diameters of at least about 5 μm, and an aerodynamic diameter of between about 1 μm and about 5 μm, preferably between about 1 μm and about 3 μm, are more capable of escaping inertial and gravitational deposition in the oropharyngeal region, and are targeted to the airways or the deep lung. The use of larger, more porous particles is advantageous since they are able to aerosolize more efficiently than smaller, denser aerosol particles such as those currently used for inhalation therapies.

In comparison to smaller particles the larger aerodynamically light particles, preferably having a VMGD of at least about 5 μm, also can potentially more successfully avoid phagocytic engulfment by alveolar macrophages and clearance from the lungs, due to size exclusion of the particles from the phagocytes' cytosolic space. Phagocytosis of particles by alveolar macrophages diminishes precipitously as particle diameter increases beyond about 3 μm. Kawaguchi, H., et al., Biomaterials 7: 61-66 (1986); Krenis, L. J. and Strauss, B., Proc. Soc. Exp. Med., 107: 748-750 (1961); and Rudt, S. and Muller, R. H., J. Contr. Rel., 22: 263-272 (1992). For particles of statistically isotropic shape, such as spheres with rough surfaces, the particle envelope volume is approximately equivalent to the volume of cytosolic space required within a macrophage for complete particle phagocytosis.

The particles may be fabricated with the appropriate material, surface roughness, diameter and tap density for localized delivery to selected regions of the respiratory tract such as the deep lung or upper or central airways. For example, higher density or larger particles may be used for upper airway delivery, or a mixture of varying sized particles in a sample, provided with the same or different therapeutic agent may be administered to target different regions of the lung in one administration. Particles having an aerodynamic diameter ranging from about 3 to about 5 μm are preferred for delivery to the central and upper airways. Particles having an aerodynamic diameter ranging from about 1 to about 3 μm are preferred for delivery to the deep lung.

Inertial impaction and gravitational settling of aerosols are predominant deposition mechanisms in the airways and acini of the lungs during normal breathing conditions. Edwards, D. A., J. Aerosol Sci., 26: 293-317 (1995). The importance of both deposition mechanisms increases in proportion to the mass of aerosols and not to particle (or envelope) volume. Since the site of aerosol deposition in the lungs is determined by the mass of the aerosol (at least for particles of mean aerodynamic diameter greater than approximately 1 μm), diminishing the tap density by increasing particle surface irregularities and particle porosity permits the delivery of larger particle envelope volumes into the lungs, all other physical parameters being equal.

The low tap density particles have a small aerodynamic diameter in comparison to the actual envelope sphere diameter. The aerodynamic diameter, dₐₑᵣ, is related to the envelope sphere diameter, d (Gonda, I., “Physico-chemical principles in aerosol delivery,” in Topics in Pharmaceutical Sciences 1991 (eds. D. J. A. Crommelin and K. K. Midha), pp. 95-117, Stuttgart: Medpharm Scientific Publishers, 1992)), by the formula:

dₐₑᵣ=d√ρ

where the envelope mass ρ is in units of g/cm³. Maximal deposition of monodispersed aerosol particles in the alveolar region of the human lung (˜60%) occurs for an aerodynamic diameter of approximately dₐₑᵣ=3 μm. Heyder, J. et al., J. Aerosol Sci., 17: 811-825 (1986). Due to their small envelope mass density, the actual diameter d of aerodynamically light particles comprising a monodisperse inhaled powder that will exhibit maximum deep-lung deposition is:

d=3/√ρ μm(where ρ<1 g/cm³);

where d is always greater than 3 μm. For example, aerodynamically light particles that display an envelope mass density, ρ=0.1 g/cm³, will exhibit a maximum deposition for particles having envelope diameters as large as 9.5 μm. The increased particle size diminishes interparticle adhesion forces. Visser, J., Powder Technology, 58: 1-10. Thus, large particle size increases efficiency of aerosolization to the deep lung for particles of low envelope mass density, in addition to contributing to lower phagocytic losses.

The aerodyanamic diameter can be calculated to provide for maximum deposition within the lungs, previously achieved by the use of very small particles of less than about five microns in diameter, preferably between about one and about three microns, which are then subject to phagocytosis. Selection of particles which have a larger diameter, but which are sufficiently light (hence the characterization “aerodynamically light”), results in an equivalent delivery to the lungs, but the larger size particles are not phagocytosed. Improved delivery can be obtained by using particles with a rough or uneven surface relative to those with a smooth surface.

In another embodiment of the invention, the particles have an envelope mass density, also referred to herein as “mass density” of less than about 0.4 g/cm³. Particles also having a mean diameter of between about 5 μm and about 30 μm are preferred. Mass density and the relationship between mass density, mean diameter and aerodynamic diameter are discussed in U.S. application Ser. No. 08/655,570, filed on May 24, 1996, which is incorporated herein by reference in its entirety. In a preferred embodiment, the aerodynamic diameter of particles having a mass density less than about 0.4 g/cm³ and a mean diameter of between about 5 μm and about 30 μm is between about 1 μm and about 5 μm.

Suitable particles can be fabricated or separated, for example by filtration or centrifugation, to provide a particle sample with a preselected size distribution. For example, greater than about 30%, 50%, 70%, or 80% of the particles in a sample can have a diameter within a selected range of at least about 5 μm. The selected range within which a certain percentage of the particles must fall may be for example, between about 5 and about 30 μm, or optimally between about 5 and about 15 μm. In one preferred embodiment, at least a portion of the particles have a diameter between about 9 and about 11 μm. Optionally, the particle sample also can be fabricated wherein at least about 90%, or optionally about 95% or about 99%, have a diameter within the selected range. The presence of the higher proportion of the aerodynamically light, larger diameter particles in the particle sample enhances the delivery of therapeutic or diagnostic agents incorporated therein to the deep lung. Large diameter particles generally mean particles having a median geometric diameter of at least about 5 μm.

In a preferred embodiment, the particles are prepared by spray drying. For example, a spray drying mixture, also referred to herein as “feed solution” or “feed mixture”, which includes the bioactive agent and one or more phospholipids selected to impart a desired or targeted release rate is fed to a spray dryer.

Suitable organic solvents that can be present in the mixture being spray dried include but are not limited to alcohols for example, ethanol, methanol, propanol, isopropanol, butanols, and others. Other organic solvents include but are not limited to perfluorocarbons, dichloromethane, chloroform, ether, ethyl acetate, methyl tert-butyl ether and others. Aqueous solvents that can be present in the feed mixture include water and buffered solutions. Both organic and aqueous solvents can be present in the spray-drying mixture fed to the spray dryer. In one embodiment, an ethanol water solvent is preferred with the ethanol:water ratio ranging from about 50:50 to about 90:10. The mixture can have a neutral, acidic or alkaline pH. Optionally, a pH buffer can be included. Preferably, the pH can range from about 3 to about 10.

The total amount of solvent or solvents being employed in the mixture being spray dried generally is greater than 99 weight percent. The amount of solids (drug, phospholipid and other ingredients) present in the mixture being spray dried generally is less than about 1.0 weight percent. Preferably, the amount of solids in the mixture being spray dried ranges from about 0.05% to about 0.5% by weight.

Using a mixture which includes an organic and an aqueous solvent in the spray drying process allows for the combination of hydrophilic and hydrophobic (i.e. phospholipids) components, while not requiring the formation of liposomes or other structures or complexes to facilitate solubilization of the combination of such components within the particles.

Suitable spray-drying techniques are described, for example, by K. Masters in “Spray Drying Handbook”, John Wiley & Sons, New York, 1984. Generally, during spray-drying, heat from a hot gas such as heated air or nitrogen is used to evaporate the solvent from droplets formed by atomizing a continuous liquid feed. Other spray-drying techniques are well known to those skilled in the art. In a preferred embodiment, a rotary atomizer is employed. An example of a suitable spray dryer using rotary atomization includes the Mobile Minor spray dryer, manufactured by Niro, Denmark. The hot gas can be, for example, air, nitrogen or argon.

Preferably, the particles of the invention are obtained by spray drying using an inlet temperature between about 100° C. and about 400° C. and an outlet temperature between about 50° C. and about 130° C.

The spray dried particles can be fabricated with a rough surface texture to reduce particle agglomeration and improve flowability of the powder. The spray-dried particle can be fabricated with features which enhance aerosolization via dry powder inhaler devices, and lead to lower deposition in the mouth, throat and inhaler device.

The particles of the invention can be employed in compositions suitable for drug delivery via the pulmonary system. For example, such compositions can include the particles and a pharmaceutically acceptable carrier for administration to a patient, preferably for administration via inhalation. The particles can be co-delivered with larger carrier particles, not including a therapeutic agent, the latter possessing mass median diameters for example in the range between about 50 μm and about 100 μm. The particles can be administered alone or in any appropriate pharmaceutically acceptable carrier, such as a liquid, for example saline, or a powder, for administration to the respiratory system.

Particles including a medicament, for example one or more of the drugs listed above, are administered to the respiratory tract of a patient in need of treatment, prophylaxis or diagnosis. Administration of particles to the respiratory system can be by means such as known in the art. For example, particles are delivered from an inhalation device. In a preferred embodiment, particles are administered via a dry powder inhaler (DPI). Metered-dose-inhalers (MDI), nebulizers or instillation techniques also can be employed.

Various suitable devices and methods of inhalation which can be used to administer particles to a patient's respiratory tract are known in the art. For example, suitable inhalers are described in U.S. Pat. No. 4,069,819, issued Aug. 5, 1976 to Valentini, et al., U.S. Pat. No. 4,995,385 issued Feb. 26, 1991 to Valentini, et al., and U.S. Pat. No. 5,997,848 issued Dec. 7, 1999 to Patton, et al. Various suitable devices and methods of inhalation which can be used to administer particles to a patient's respiratory tract are known in the art. For example, suitable inhalers are described in U.S. Pat. Nos. 4,995,385, and 4,069,819 issued to Valentini, et al., U.S. Pat. No. 5,997,848 issued to Patton. Other examples include, but are not limited to, the Spinhaler® (Fisons, Loughborough, U.K.), Rotahaler® (Glaxo-Wellcome, Research Triangle Technology Park, North Carolina), FlowCaps® (Hovione, Loures, Portugal), Inhalator® (Boehringer-Ingelheim, Germany), and the Aerolizer® (Novartis, Switzerland), the diskhaler (Glaxo-Wellcome, RTP, NC) and others, such as known to those skilled in the art. Preferably, the particles are administered as a dry powder via a dry powder inhaler.

Preferably, particles administered to the respiratory tract travel through the upper airways (oropharynx and larynx), the lower airways which include the trachea followed by bifurcations into the bronchi and bronchioli and through the terminal bronchioli which in turn divide into respiratory bronchioli leading then to the ultimate respiratory zone, the alveoli or the deep lung. In a preferred embodiment of the invention, most of the mass of particles deposits in the deep lung. In another embodiment of the invention, delivery is primarily to the central airways. Delivery to the upper airways can also be obtained.

In one embodiment of the invention, delivery to the pulmonary system of particles is in a single, breath-actuated step, as described in U.S. patent application, High Efficient Delivery of a Large Therapeutic Mass Aerosol, application Ser. No. 09/591,307, filed Jun. 9, 2000, Attorney Docket No. 2685.2001-000, which is incorporated herein by reference in its entirety. In another embodiment of the invention, at least 50% of the mass of the particles stored in the inhaler receptacle is delivered to a subject's respiratory system in a single, breath-activated step. In a further embodiment, at least 5 milligrams and preferably at least 10 milligrams of a medicament is delivered by administering, in a single breath, to a subject's respiratory tract particles enclosed in the receptacle. Amounts as high as 15, 20, 25, 30, 35, 40 and 50 milligrams can be delivered.

As used herein, the term “effective amount” means the amount needed to achieve the desired therapeutic or diagnostic effect or efficacy. The actual effective amounts of drug can vary according to the specific drug or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations, (e.g. by means of an appropriate, conventional pharmacological protocol). For example, effective amounts of albuterol sulfate range from about 100 micrograms (μg) to about 10 milligrams (mg).

Aerosol dosage, formulations and delivery systems also may be selected for a particular therapeutic application, as described, for example, in Gonda, I. “Aerosols for delivery of therapeutic and diagnostic agents to the respiratory tract,” in Critical Reviews in Therapeutic Drug Carrier Systems, 6: 273-313, 1990; and in Moren, “Aerosol dosage forms and formulations,” in: Aerosols in Medicine. Principles, Diagnosis and Therapy, Moren, et al., Eds, Esevier, Amsterdam, 1985.

The particles of the invention have a homogeneous dispersion of drug throughout the particles rather than liposome or liposome forming characteristics. Without wishing to be held to any particular interpretation of the mechanism of the invention, it is believed that large porous particles, also referred to herein as aerodynamically light particles, intended for delivery of drugs to the lungs encounter several different environmental conditions (i.e., temperature and humidity) during their lifetime. Once spray-dried, these particles are generally packaged and stored at room temperature. Upon delivery to humans, the particles encounter various conditions en route to the deep parts of the lungs. During transit through the bronchi, the particles are carried in inspired air which quickly becomes warmed to body temperatures and saturated with water (˜100% humidity at 37° C.). Once in the alveolar region, the particles may encounter regions with (a) thin layers of water (less than 1 micron) and (b) deeper pools of water (greater than microns in depth), both of which are covered by lung surfactant. The alveolar regions also contain macrophages, which attempt to engulf and remove foreign particles. The particle integrity and potential for sustained release of the particles depend in part on the ability of the particles to remain intact upon encountering these varying environmental conditions.

The nature of the lipid used is believed to play a major role in the physical integrity of the particles. For example, in the bulk hydrated state, DPPC has a gel to liquid-crystalline transition temperature (T_{c}) of approximately 41° C. Below this temperature, bulk hydrated DPPC molecules exist in either crystalline or rigid gel forms, with their hydrocarbon chains closely packed together in an ordered state. Above this temperature, the hydrocarbon chains of DPPC expand and become disordered, and become easier to disrupt. Increasing the hydrocarbon chain lengths of a saturated phosphatidylcholine by two units each results in an increase in this transition temperature. For example, distearoylphosphatidylcholine (DSPC) has a T_{c} of approximately 55° C. an increase of 14° C. compared to that of DPPC. Additionally, other types of phospholipids having different-head groups can have higher transition temperatures than phosphatidylcholines for the same hydrocarbon chain lengths; for example, dipalmitoyl-phosphatidylethanolamine (DPPE) has a T_{c} of approximately 63° C. an increase of 22° C. compared to that of DPPC. Phospholipids such as these will tend to exist in a more rigid form in the bulk state as compared to DPPC at a given temperature. As a result, the lipid composition of a large porous particles for pulmonary drug delivery potentially has a significant influence on the particle matrix transition which could be important for the particle physical integrity and their sustained release properties.

The present invention will be further understood by reference to the following non-limiting examples.

Exemplification

Geometric size distributions were determined using a Coulter Multisizer II. Approximately 5-10 mg of powder was added to 50 mL isoton II solution until the coincidence of particles was between 5 and 8%. Greater than 500,000 particles were counted for each batch.

Aerodynamic size distribution was determined using an Aerosizer/Aerodispenser (Amherst Process Instruments, Amherst, Mass.). Approximately 2 mg powder was introduced into the Aerodisperser and the aerodynamic size was determined by time of flight measurements.

### EXAMPLE 1A

To test the dependence of drug release on the transition temperature of the particle matrix, powders containing phospholipid and the small hydrophilic drug albuterol sulfate were spray-dried. A 70% anhydrous ethanol and 30% distilled water solvent was employed. Table 3 shows the composition of the particles:

TABLE 3

| | DPPC† | DSPC‡ | L-Leucine | Albuterol Sulfate |
|---|---|---|---|---|
| Formulations | (% w/w) | (% w/w) | (% w/w) | (% w/w) |
| A | 66 | 0 | 17 | 17 |
| B | 33 | 33 | 17 | 17 |
| C | 0 | 66 | 17 | 17 |

†1,2-Dipalmitoyl-sn-glycero-3-phosphocholine
‡1,2-Distearoyl-sn-glycero-3-phosphocholine

In vitro release experiments were performed using phosphate buffered saline (PBS; 10 mM, pH 7.4) as the dissolution medium. Unformulated albuterol sulfate or albuterol sulfate dry powder formulations were deposited on filter membranes using a filter holder and a vacuum pump operated at 60 L/min. Polyvinyldiene fluoride (PVDF) membrane filters (0.45 μm porosity) were used in this study. All dissolution experiments were carried out at 37° C. using a flow through dissolution apparatus. Using this apparatus, the dissolution medium was circulated by means of a peristaltic pump at 10 ml/min flow rate past the filter. Samples were withdrawn from the dissolution medium reservoir at predetermined time points. Withdrawn sample volume was replenished by adding equal volume of fresh buffer in the medium reservoir. Samples were analyzed by monitoring UV absorbance at 280 nm. The cumulative amount of albuterol sulfate dissolved was expressed as a percentage of the initial total albuterol sulfate deposited on the filter and plotted against time. Dissolution profiles were fitted to the first order release equation:

C₍ₜ₎=C_{(inf)}*(1−e^{-k*t})

where, k is the first order release constant, C₍ₜ₎ is the concentration of albuterol sulfate at time t (min) and C_{(inf)} is the maximal theoretical albuterol sulfate concentration in the dissolution medium.

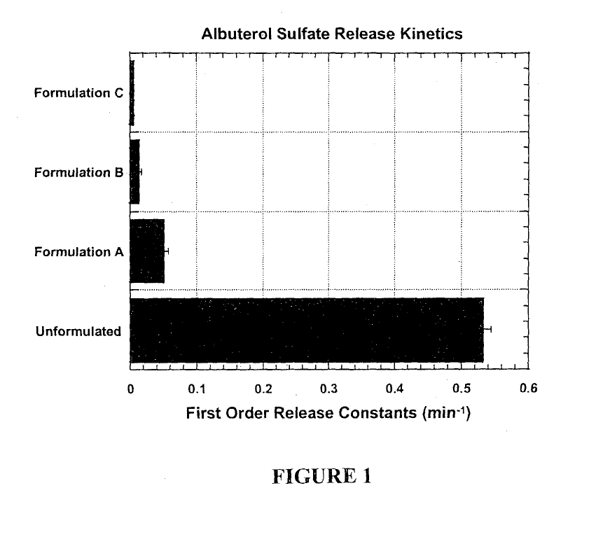

FIG. 1 shows the first order release constants for the three different formulations (A, B and C). The release rate was slowest for dry powder formulation C with the phospholipid having the higher transition temperature (DSPC; theoretical transition at 55° C.) and fastest for dry powder formulation A with the phospholipid having the lower transition temperature (DPPC; theoretical transition at 41° C.). Dry powder formulation B, with a combination of DPPC and DSPC, showed an intermediate release rate.

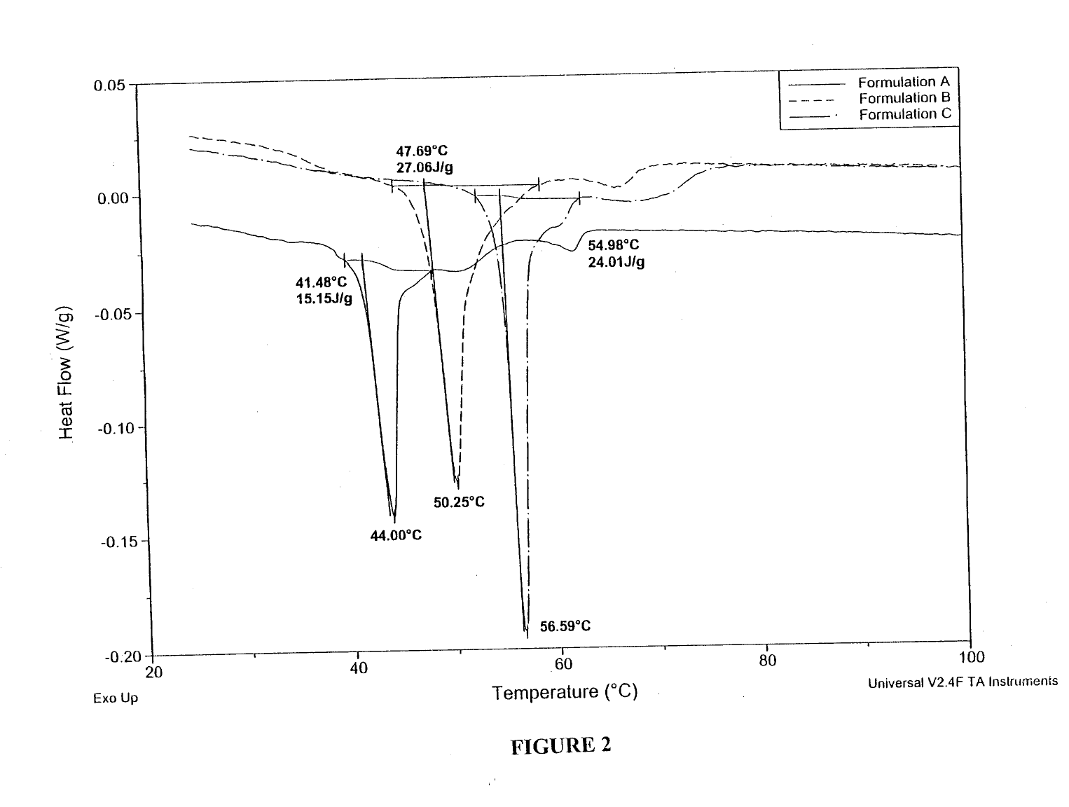

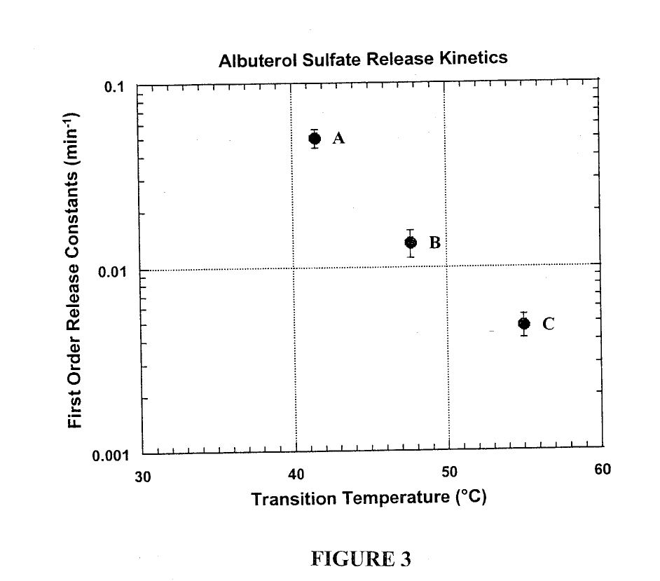

Differential scanning calorimetry (DSC) measurements (heating rate of 1° C./min) of formulations A, B and C were performed. The thermograms are shown in FIG. 2. Results from these experiments showed that the formulation having the highest matrix transition temperature caused the slowest rate of release and vice versa. The inverse relationships between matrix transition temperature and the first order release constants are shown in FIG. 3.

### EXAMPLE 1B

To test if proteins could be formulated with excipients having high and low transition temperature powders containing phospholipid and a model protein, human serum albumin (HSA), were spray-dried using a 70% anhydrous ethanol and 30% distilled water solvent. The compositions of particles are presented in Table 4.

TABLE 4

| | DPPC | DSPC | Albumin |
|---|---|---|---|
| Formulation | (% w/w) | (% w/w) | (% w/w) |
| I | 80 | 0 | 20 |
| II | 0 | 80 | 20 |

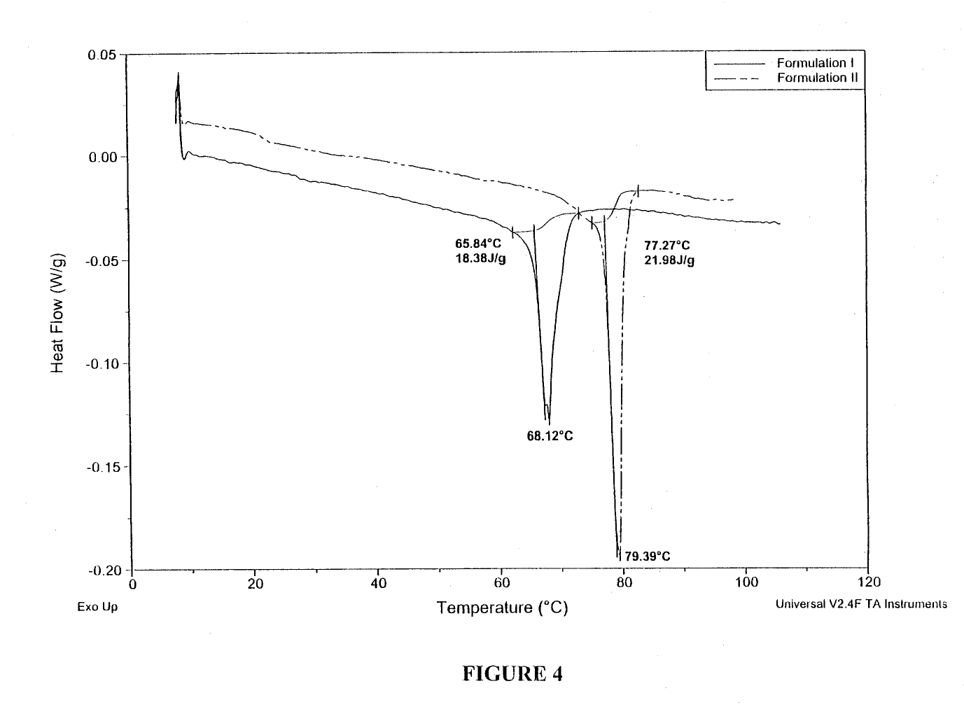

Thermograms from DSC experiments are shown in FIG. 4. Matrix transition temperature for particles formulated with DPPC (Formulation I) was lower than that for particles formulated with DSPC (Formulation II). The results showed that the matrix transition temperature for particles also can be controlled for particles including macromolecules for example human serum albumin by choosing appropriate components. These results also demonstrated that small molecules as well as peptides/proteins may be used in particles having different matrix transition temperatures.

### EXAMPLE 2

Particles containing albuterol sulfate were prepared as already described above. The spray-drying parameters were inlet temperature 143° C., feed rate 100 ml/min, atomization speed 47000 RPM, and process air, 92 kg/hr.

Table 5 illustrates the compositions, tap density, mass median geometric diameter (MMGD) and the mass median aerodynamic diameter (MMAD) of several batches of particles.

The results illustrate that the particles are suitable for delivery to the pulmonary system, in particular to the deep lung.

TABLE 5

| For- | | | Albuterol | | | Tap |
|---|---|---|---|---|---|---|
| mu- | DSPC* | L-Leucine | Sulfate | MMAD | MMGD | Density |
| lations | (% w/w) | (% w/w) | (% w/w) | (μm) | (μm) | (g/c.c) |
| 1a | 60 | 36 | 4 | 2.783 | 8.226 | 0.11 |
| 1b | 60 | 36 | 4 | 2.379 | 10.28 | 0.05 |
| 1c | 60 | 36 | 4 | 2.661 | 8.083 | 0.11 |
| 2a | 76 | 20 | 4 | 3.068 | 10.530 | 0.09 |
| 2b | 76 | 20 | 4 | 3.232 | 11.760 | 0.08 |

*1,2-Distearoyl-sn-glycero-3-phosphocholine

### EXAMPLE 3

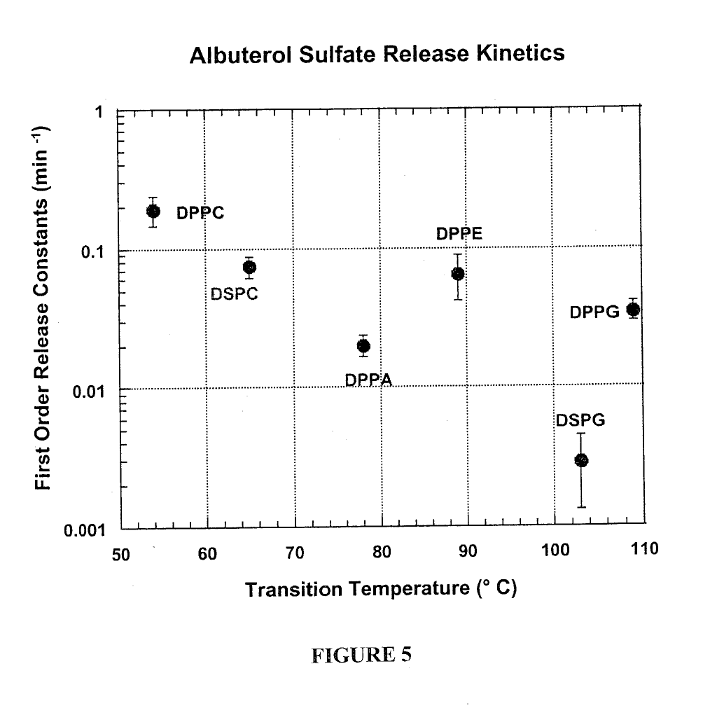

Particles containing albuterol sulfate were prepared as described above. The formulations (76% phospholipid, 20% leucine and 4% albuterol sulfate) were spray dryed from a 70/30 (v/v) ethanol/water solvent. In vitro release and DSC was performed as described above. The composition and results for different formulations are shown in Table 6. FIG. 5 is a plot showing the correlation between the first order release constants and matrix transition temperature for different albuterol sulfate dry powder formulations.

TABLE 6

| | | Powder | |
|---|---|---|---|
| | | Matrix | |
| | | Transition | |
| | Phospholipids | Temperatures | First Order Release |
| Formulations | (76% w/w)† | (° C.)‡ | Constants (min⁻¹) |
| i | DPPC | 54 | 0.1916 ± 0.0408 |
| ii | DSPC | 65 | 0.0739 ± 0.0109 |
| iii | DPPA | 78 | 0.0199 ± 0.0027 |
| iv | DPPE | 89 | 0.0643 ± 0.0211 |
| v | DPPG | 109 | 0.0348 ± 0.0045 |
| vi | DSPG | 103 | 0.0029 ± 0.0015 |

†20% w/w L-leucine and 4% w/w albuterol sulfate.
‡as calculated by DSC

### EXAMPLE 4

The purpose of this study was to determine the influence of the transition temperatures of the material used to make the particles on the physical integrity of the particles under fully hydrated conditions. The study was designed to assess the integrity of large porous blank particles, e.g., particles which do not include a bioactive agent, under in vitro environmental conditions. The study was carried out to determine the integrity of particles in bulk water environments. A Coulter Multisizer was employed to monitor the changes in the geometric size of the particles as a function of time in a saline solution at both 25 and 37° C. Optical microscopy was used to examine the morphology of the particles as a function of time in conjunction with the Coulter Multisizer measurements.

The formulations used to test the effects of using phospholipids with higher chain melting transition temperatures than DPPC due to either headgroup or acyl chain on the integrity of the particles in bulk water environments are shown in Table 7.

TABLE 7

| | | MMAD | | Calculated |
|---|---|---|---|---|
| | Compositions | § | VMGD† | Density |
| Formulations | (% w/w) | (μm) | (μm) | (g/cc)‡ |
| A | 70:20:10 DPPC:Sodium | 2.10 | 10.0 | 0.04 |
| | Citrate:Calcium Chloride | | | |
| B | 60:20:20 DPPC:Human | 3.84 | 7.32 | 0.28 |
| | serum | | | |
| | albumin:Lactose | | | |
| C | 35:35:20:10 | 3.87 | 7.35 | 0.28 |
| | DPPC:DSPC: | | | |
| | Sodium Citrate:Calcium | | | |
| | Chloride | | | |
| D | 70:30 DSPC:Leucine | 3.64 | 7.20 | 0.26 |
| E | 60:40 DPPE:Leucine | 4.46 | 9.53 | 0.22 |

§Mass median aerodynamic diameter
†Volumetric median geometric diameter
‡Based on the equation dₐₑᵣ = d_{g}√ρ

The changes in the morphology of particles upon addition of bulk water were examined via optical microscopy. First, the particles were dispersed onto a dry microscope slide and subsequently imaged in the dry state. Next, a droplet of water at 25° C. was placed on the slide, and the morphology of the particles suspended in the water droplet was recorded. Images were taken until the droplet was completely evaporated (which typically would occur after a time period of approximately ten minutes).

The size and morphology of the particle formulations were monitored as a function of time at 25 and 37° C. via the following procedure:

i. Approximately 2 mg of particles were placed in 15 ml of isotone (a physiologically-based medium consisting of filtered buffered saline) maintained at either 25 or 37° C. and slowly stirred.

ii. At selected time points, 200 microliters of the suspension from step (i) was placed in 20 ml of isotone and analyzed for particle size content using a Coulter Multisizer.

iii. Concurrent with step (ii), a droplet of the solution from step (i) was placed onto a microscope slide and particles suspended in the droplet were imaged using an optical microscope.

The results show that particles containing DPPC maintained their physical integrity in bulk water at 25° C. (Table 8), but began to lose their relativly large particle geometric diameter at 37° C. (Table 8). In contrast, particles containing phospholipids such as DSPC and DPPE appeared to maintain their physical integrity in bulk water at 37° C. (Table 9). These results indicated that formulations containing DSPC and DPPE appear to maintain their physical integrity under fully hydrated conditions and thus have the potential to be used in sustained release of drug molecules when delivered to the lungs.

The results obtained indicated that the lipid composition of the blank particles greatly influences and can be used to control the physical integrity and dissolution rate of the particles under bulk water conditions.

TABLE 8

| | Particle Geometric Diameter in μm at Time | | | | | |
|---|---|---|---|---|---|---|
| Formulations | 0 min | 15 min | 30 min | 1 hr | 2 hr | 4 hr |
| A | 9.15 | 9.61 | 9.77 | 9.91 | 10.2 | 10.9 |
| B | 7.47 | 7.83 | 8.03 | 8.44 | 8.78 | 9.73 |
| C | 7.03 | 7.55 | 7.60 | 7.64 | 7.68 | 7.55 |
| D | 6.79 | 8.36 | 8.03 | 8.34 | 8.61 | 8.63 |
| E | 8.63 | 8.65 | 8.67 | 8.64 | 9.44 | 9.34 |

Particle dissolution vs time at 25° C.

TABLE 9

| | Particle Geometric Diameter in μm at Time | | | | | | |
|---|---|---|---|---|---|---|---|
| Formulations | 0 min | 15 min | 30 min | 1 hr | 2 hr | 4 hr | 24 hr |
| A | 9.72 | * | * | * | * | * | — |
| B | 8.13 | 2.35 | — | — | — | — | — |
| C | 7.92 | 8.19 | 8.29 | 7.98 | 7.78 | 7.83 | 7.49 |
| D | 8.08 | 8.25 | 8.29 | 8.43 | 8.39 | 8.52 | 8.32 |
| E | 8.69 | ND | 9.09 | 9.13 | 9.57 | 10.2 | — |

Particle dissolution vs time at 37° C.
* Loss of primary particle peak.
— Absence of detectable particle peak.
ND: Not determined.

### EXAMPLE 5

Particles containing albuterol sulfate were prepared as described, having a composition of 76% DSPC, 20% leucine and 4% albuterol sulfate (Formulation A) or 60% DPPC, 36% leucine and 4% albuterol sulfate (Formulation B). Their properties are shown in Table 10.

TABLE 10

| | | | Calculated |
|---|---|---|---|
| | MMAD§ | VMGD† | Density |
| Formulations | (μm) | (μm) | (g/cc)‡ |
| A | 3.4 | 10.6 | 0.10 |
| B | 2.9 | 9.8 | 0.09 |

§Mass median aerodynamic diameter
†Volumetric median geometric diameter
‡Based on the equation dₐₑᵣ = d_{g}√ρ

Male Hartley guinea pigs were obtained from Hilltop Lab Animals (Scottsdale, Pa.). At the time of use, the animals weighed between 389 and 703 g and were approximately 60 to 90 days old. The animals were in good health upon arrival and remained so until use; no clinical signs of illness were observed at any time. The animals were housed one animal to a cage in AAALAC approved standard plastic cages placed in cubicles; each cubicle could accommodate up to 25 cages. At least one sentry guinea pig was maintained in each cubicle. The bedding used in the cages was Alphachip heat treated pine softwood laboratory bedding (Northeastern Products Corp., Warrensburg, N.Y.). Since the animals were housed individually, they were identified by writing the animal number on the cage card. The animals were allowed to acclimate to their surroundings for at least one week prior to use. The animals were housed for no more than 1 month before use. The light/dark cycle was 12/12 hours. The temperature in the animal room was ambient room temperature of approximately 70° F. The animals were allowed free access to food and water. The food was Lab Diet-Guinea Pig #5025 (PMI Nutrition International, Inc., Brentwood, Mo.). The water was from a clean tap source.

A dose of 5 mg of powder (the amount of powder necessary to deliver 200 μg of albuterol sulfate) was administered via forced inhalation. Each dose was weighed gravimetrically into 100 mL pipette tips. Briefly, the pointed end of the pipette tip was sealed with parafilm, the appropriate amount of powder was placed into the pipette tip and weighted. After an appropriate amount of powder was contained in the pipette tip, the large end of the pipette tip was sealed with parafilm. The doses were stored vertically (with the small tip end down) in scintillation vials that were then placed in plastic boxes containing dessicant and stored at room temperature. Before weighing, the bulk powders are stored in a dry room with controlled temperature and humidity. The doses were based on % w/w. The dose of drug used in all of the studies was 200 μg of albuterol sulfate. Since each powder used was 4% w/w albuterol sulfate, the total weight of powder administered per dose was 5 mg. There was no modification of the dose based on weight. Animals were anesthetized with 60 mg/kg of ketamine and 2 mg/kg of xylazine delivered i.p. Guinea pigs were then tracheotomized with a small hard tip cannula. The powder was delivered via a ventilator set at 4 ml air volume and a frequency of 60 breaths/min. After powder delivery, the guinea pig throat was closed with wound clips. Guinea pigs were then returned to his cage until lung resistance was assessed. For more detail in the forced inhalation maneuver, see Ben-Jebria A, et al., Pharm Res 1999 16(4):555-61. The dose was administered only once in each animal.

The endpoint in this study was pharmacodynamic. Albuterol sulfate was administered at a given time before challenge with a known bronchoconstrictor, carbachol. The equipment used for determination of lung resistance is from Buxco Electronics. The Buxco system uses changes in pressure and flow within a plethysmograph to determine lung resistance to airflow. To correct for variations in baseline resistance, the change in lung resistance (ΔRL) is reported. Therefore, as the change in lung resistance increases, the animal is increasingly bronchoconstricted.

Each guinea pig was anesthetized with 60 mg/kg of ketamine and 2 mg/kg of xylazine delivered i.p. A tracheal cannula was inserted into the trachea and firmly tied in place using suture. The animal was then placed into the plethysmograph and the tracheal cannula was attached to a port that is connected to a transducer. Succinylcholine (5 mg/kg) injected i.p. is administered to eliminate spontaneous breathing. Once spontaneous breathing was stopped, the animal was ventilated (4ml, 60 breaths/min) for the remainder of the experiment. The Buxco program was then started. After 7 minutes of stabilization, the plythesmograph was opened and carbachol (130 μg/kg) was administered i.p. The data collection period was then conducted for a total of 60 min. Mean lung resistanc (RL) is determined for 0-2, 10-15, 30-35 and 55-60 min. The change in RL is determined by subtracting the lowest mean RL (usually at either 0-2 or 10-15 min) from the highest mean RL (usually at 55-60 min). For more information, see Ben-Jebria A, et al., Pharm Res 1999 16(4):555-61.

Animals were assigned to one of three treatment groups: Formulation A, Formulation B and placebo. After collecting all the 15-16 hour data for each group, animals were then dosed and data collected at the following time points in this order: 24 hours, 30-60 min and 20-21 hours post dose.

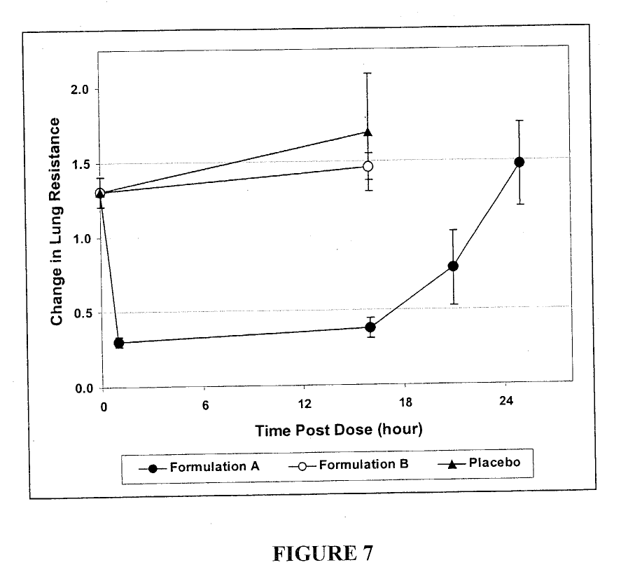

Intratracheal administration of Formulation A, using forced inhalation, reduced the ability of carbachol to induce increased lung resistance. The protective effect of Formulation A was apparent by 30-60 minutes and lasted up to 20-21 hours (FIG. 7). In addition, for comparison the pharmacodynamic effects of formulations A and B at 15-16 hour post dosing are shown in the Table 11. These data showed that the duration of the pharmacodynamic effect of albuterol sulfate formulations was dependent on the excipients in that particles having higher matrix transition (e.g., DSPC; Formulation A) provided prolonged protection against carbachol compared to particles having lower matrix transition (e.g., DPPC; Formulation B).

TABLE 11

| Formulations | ΔRL (mean ±SEM) § |
|---|---|
| Placebo | 1.307 ± 0.0100 |
| A | 0.3790 ± 0.0671 |
| B | 1.459 ± 0.0905 |

§ The ΔRL (change in lung resistance) values were determined at 15-16 hours post dose.

While this invention has been particularly shown and described with references to preferred embodiments thereof, it will be understood by those skilled in the art that various changes in form and details may be made therein without departing from the scope of the invention encompassed by the appended claims.

What is claimed is:

1. Particles for modulation of drug release comprising:
(a) a bioactive agent; and
(b) a phospholipid or a combination of phospholipids, said phospholipid or combination of phospholipids, said particles having in a matrix transition temperature corresponding to a targeted release rate of the biologically active agent from the particles and a tap density of less than about 0.4 g/cm³.

2. The particles of claim 1 wherein the particles have a tap density less than about 0.1 g/cm³.

3. The particles of claim 1 wherein the particles have a mean geometric diameter of between about 5 microns and about 30 microns.

4. The particles of claim 3 wherein the particles have a mean geometric diameter of between about 9 microns and 30 microns.

5. The particles of claim 1 wherein the particles have an aerodynamic diameter of between about 1 micron and about 5 microns.

6. The particles of claim 5 wherein the particles have an aerodynamic diameter of between about 1 micron and 3 microns.

7. The particles of claim 5 wherein the particles have an aerodynamic diameter of between about 3 microns and 5 microns.

8. The particles of claim 1 further comprising a compound selected from the group consisting of polysaccharides, sugars, amino acids, polymers, proteins, lipids, surfactants, cholesterol, fatty acids, fatty acid esters and any combination thereof.

9. The particles of claim 1 wherein the bioactive agent is present in the particles in an amount of at least 0.1% weight.

10. The particles of claim 1 wherein the bioactive agent is albuterol sulfate or estrone sulfate.

11. The particles of claim 1 wherein the bioactive agent is a protein or peptide.

12. The particles of claim 1 wherein the bioactive agent is hydrophilic.

13. The particles of claim 1 wherein the bioactive agent is hydrophobic.

14. The particles of claim 1 wherein the phospholipid or the combination of phospholipids is present in the particles in an amount of between about 5 and about 99 weight %.

15. The particles of claim 1 wherein the matrix transition temperature is less than or equal to a subject's physiological temperature.

16. The particles of claim 1 wherein the transition temperature is higher than a subject's physiological temperature.

17. A method comprising delivering via the pulmonary system of a patient in need of treatment, prophylaxis or diagnosis an effective amount of the particles of claim 1.

18. A method for delivery via the pulmonary system comprising administering to the respiratory tract of a patient in need of treatment, prophylaxis or diagnosis an effective amount of particles having a selected release rate of a bioactive agent, said particles comprising:
(a) the bioactive agent; and
(b) a phospholipid or a combination of phospholipids, said phospholipid or combination of phospholipids;
wherein the particles have a matrix transition temperature corresponding to a targeted release rate of the therapeutic, prophylactic or diagnostic agent from the particles and a tap density of less than about 0.4 g/cm³.

19. The method of claim 18 wherein the particles have a tap density less than about 0.1 g/cm³.

20. The method of claim 18 wherein the particles have a mean geometric diameter of between about 5 microns and about 30 microns.

21. The method of claim 18 wherein the particles have a mean geometric diameter of between about 10 microns and 30 microns.

22. The method of claim 18 wherein the particles have an aerodynamic diameter of between about 1 and 5 microns.

23. The method of claim 22 wherein the particles have an aerodynamic diameter of between about 1 micron and about 3 microns.

24. The method of claim 22 wherein the particles have an aerodynamic diameter of between about 3 microns and about 5 microns.

25. The method of claim 18 wherein delivery is primarily to the deep lung.

26. The method of claim 18 wherein delivery is primarily to the central airways.

27. The method of claim 18 wherein delivery is primarily to the upper airways.

28. The method of claim 18 wherein the particles further comprise a compound selected from the group consisting of polysaccharides, sugars, amino acids, polymers, lipids, surfactants, cholesterol, fatty acids, fatty acid esters, proteins, peptides cyclodextrins, surfactants and and any combination thereof.

29. The method of claim 18 wherein the bioactive agent is present in the particles in an amount of at least 0.1 weight %.

30. The method of claim 18 wherein the bioactive agent is selected from the group consisting of albuterol sulfate or estrone sulfate.

31. The method of claim 18 wherein the bioactive agent is a protein or peptide.

32. The method of claim 18 wherein the bioactive agent is hydrophilic.

33. The method of claim 18 wherein the bioactive agent is hydrophobic.

34. The method of claim 18 wherein the phospholipid or the combination of phospholipids is present in the particles in an amount of between about 5 and about 99 weight %.

35. The method of claim 18 wherein the matrix transition temperature is less than or equal to a subject's physiological temperature.

36. The method of claim 18 wherein the transition temperature is higher than a subject's physiological temperature.

37. The method of claim 18 wherein administration is via a dry powder inhaler.

38. A method for delivery via the pulmonary system particles having a release rate from the particles of a therapeutic, prophylactic or diagnostic agent comprising:
administering to the respiratory system of a patient in need of treatment, prophylaxis or diagnosis an effective amount of particles comprising:
(a) the therapeutic, prophylactic or diagnostic agent, or combinations thereof; and
(b) a phospholipid or a combination of phospholipids, said phospholipid or combination of phospholipids resulting in a matrix transition temperature such that the particles have the release rate;
wherein the particles have a tap density less than about 0.4 g/cm³.

39. A method for increasing a release time of a therapeutic, prophylactic or diagnostic agent comprising administering to a patient in need of treatment, prophylaxis or diagnosis an effective amount of particles comprising:
(a) a therapeutic, prophylactic or diagnostic agent; and
(b) a phospholipid or a combination of phospholipids, said phospholipid or combination of phospholipids;
wherein the particles have a transition temperature higher than the physiological temperature of the patient and a tap density of less than about 0.4 g/cm³.

40. Particles for modulation of drug release having a tap density of less than about 0.4 g/cm³ comprising:
(a) a therapeutic, prophylactic or diagnostic agent; and
(b) a phospholipid or combination of phospholipids, said phospholipid or combination of phospholipids having a transition temperature higher than the body temperature of a human or veterinary subject.
